(19) United States
(12) Patent Application Publication
(10) Pub. No.: US 2014/0128759 A1
(43) Pub. Date: May 8, 2014

(54) Monitoring Apparatus and Method

(71) Applicant: Spektikor Oy, Oulu (FI)

(72) Inventor: Ilkka Heikkilä, Oulu (FI)

(73) Assignee: Spektikor Oy, Oulu (FI)

(21) Appl. No.: 14/045,566

(22) Filed: Oct. 3, 2013

(30) Foreign Application Priority Data
Nov. 6, 2012 (GB) 1219930.3

(51) Int. Cl. A61B 5/00; A61B 5/0245

(52) U.S. Cl. CPC A61B 5/7217; A61B 5/0245
USPC 600/519

(57) ABSTRACT

A monitoring apparatus (50) monitors heart activity by sensing one or more signals accessible at an outer skin surface of a person (100). The monitoring apparatus (50) includes one or more electrodes for contacting onto the outer skin surface of the person (100), a signal processing arrangement (108) for processing one or more signals provided from the one or more electrodes to generate corresponding processed signals, and a display arrangement (106) for receiving the processed signals to generate presentation information from which a heart beat rate of the person (100) is discernible. The signal processing arrangement includes a detector for detecting signal artefacts arising from one or more electronic devices coupled to the person (100) and for removing an influence of the signal artefacts from the processed signals, such that the processed signals provide a more accurate indication of the heart beat rate. Optionally, the signal processing arrangement is operable to remove signal artefacts arising from the one or more electronic devices coupled to the person (100) being a pacemaker. The apparatus (50) is thus capable of providing more accurate monitoring of heart beat rate.

### FIELD OF THE INVENTION

The present invention relates to monitoring apparatus for detecting heartbeat using electrocardiography (ECG), for example the invention concerns monitoring apparatus for detecting heartbeat using electrocardiography (ECG), wherein the apparatus includes a filtering arrangement for processing signals sensed by the apparatus for reducing artefacts therein caused by electronic devices such as pacemakers to provide a more representative indication of heart function. Moreover, the present invention concerns methods of detecting heartbeat using electrocardiography (ECG), for example to methods of detecting heartbeat using electrocardiography (ECG), wherein the method includes using a filtering arrangement for processing sensed signals for reducing artefacts therein caused by electronic devices such as pacemakers to provide a more representative indication of heart function. Furthermore, the invention relates to software products stored on a machine-readable data carrier and executable upon computing hardware for implementing aforesaid methods.

### BACKGROUND OF THE INVENTION

Electrocardiography (ECG) is a technique for measuring and representing a temporal electrical activity of a heart, wherein associated electrical signals are measured using electrodes attached to an outer skin surface of a given person including the heart. Such measurement is a non-invasive procedure. An ECG is customarily displayed on a computer screen or printed onto paper for viewing. Moreover, an ECG provides test records of electrical heart activity and is frequently used to measure a rate and a regularity of heartbeats. An ECG may be employed, for example in a hospital environment, for measuring and diagnosing abnormal heart beat rhythms.

An apparatus for performing ECG is operable to detect and amplify tiny electrical changes, for example in an order of milliVolts (mV) amplitude, on an outer skin surface of a given person, wherein the tiny electrical changes are caused when a heart muscle of the given person depolarizes during each heartbeat. At rest, each heart muscle cell has a negative charge across its cell membrane. When this negative charge decreases towards zero, via an influx of positive cations, namely Na+ and Ca++ cations, depolarization occurs, which activates mechanisms in the cell that cause it to contract. During each heartbeat, a healthy heart will exhibit an orderly progression of a wave of electrical depolarisation across its spatial extent. This wave is detected as tiny rises and falls in a voltage developed between two electrodes placed on the outer skin surface, spatially either side of the heart; the developed voltage sensed by the two electrodes is amplified and then displayed as a wavy line either on a screen or recorded on paper. This wavy line indicates an overall rhythm of the heart, wherein weaknesses in different parts of the heart muscle can be determined from inspection of the wavy line. An irregular heart pattern represented on the wavy line of an ECG is known as an arrhythmia.

Usually, more than two electrodes are used when sensing voltages for generating an ECG; conveniently, sensed voltages are combined into a number of pairs. The output from each pair is known as a lead. Each lead provides a representation of heart function from a different angle relative to an elongate axis of a body of the given person. Different types of ECG's are conveniently referred to by the number of leads that are recorded, for example “3-lead”, “5-lead” or “12-lead” ECG's. A 12-lead ECG is one in which twelve different electrical signals are recorded at approximately the same time and will often be used as a one-off recording of an ECG, contemporarily printed out as a paper copy. Conversely, 3-lead and 5-lead ECG's tend to be monitored continuously and viewed only on a screen of an appropriate monitoring device, for example during an operation or whilst a given person is being transported in an ambulance. Optionally, a permanent record of a 3- or 5-lead ECG may be taken, depending upon equipment used.

Contemporary ECG apparatus, for example ECG monitors, include multiple filters for processing measured heart signals sensed by skin outer surface electrodes. Most common settings for such apparatus are a monitor mode and a diagnostic mode. In the monitor mode, a low-frequency filter, for low-pass filtering the measured heart signals sensed by the skin outer surface electrodes, is set at either 0.5 Hz or 1 Hz and a high-frequency filter, for low-pass filtering the measured heart signals sensed by the skin outer surface electrodes, is set at 40 Hz. Such filtering limits signal artefacts for routine cardiac rhythm monitoring. The high-pass filter helps reduce a wandering baseline component of the measured heart signals, and the low-pass filter helps reduce 50- or 60-Hz power line noise components present in the measured heart signals; the power line network frequency differs between 50 and 60 Hz in different countries. In the diagnostic mode, the high-pass filter is set at 0.05 Hz, which allows accurate ST segments to be recorded. The high-pass filter is set to 40, 100, or 150 Hz. Consequently, the monitor mode ECG display is more filtered than diagnostic mode, because its passband is narrower.

A problem encountered in practice is that such high-pass and low-pass filters are not able to remove artefacts caused by pacemakers and similar heart-stimulating electronic devices which may contribute to the aforementioned measured heart signal. In emergency situations, when it is important to determine whether or not heart functioning has ceased, for example by inexperienced personnel, such artefacts can result in incorrect assessments being made and associated inappropriate treatment being administered.

### SUMMARY OF THE INVENTION

The present invention seeks to provide a monitoring apparatus which is more reliably capable of monitoring heart operation and providing monitoring results, even when artefacts caused by pacemakers and similar heart-stimulating electronic devices are contributing to measured heart signals.

Moreover, the present invention seeks to provide an improved method of monitoring heart operation and providing monitoring results, even when artefacts caused by pacemakers and similar heart-stimulating electronic devices are contributing to measured heart signals.

According to a first aspect of the invention, there is provided a monitoring apparatus as claimed in appended claim 1: there is provided a monitoring apparatus for monitoring heart activity by sensing one or more signals accessible at an outer skin surface of a person, wherein the monitoring apparatus includes one or more electrodes for contacting onto the outer skin surface of the person, a signal processing arrangement for processing one or more signals provided from the one or more electrodes to generate corresponding processed signals, and a display arrangement for receiving the processed signals to generate presentation information from which a heart beat rate of the person is discernible, characterized in that the signal processing arrangement includes a detector for detecting signal artefacts arising from one or more electronic devices coupled to the person and for removing an influence of the signal artefacts from the processed signals, such that the processed signals provide a more accurate indication of the heart beat rate.

The invention is of advantage in that detection and removal of artefacts arising from the one or more electronic devices coupled to the person and for removing an influence of the signal artefacts from the processed signals enables a more accurate indication of the heart beat rate to be achieved.

Optionally, in the monitoring apparatus, the signal processing arrangement is operable to remove signal artefacts arising from the one or more electronic devices coupled to the person being a pacemaker.

More optionally, in the monitoring apparatus, the signal processing arrangement is operable to insert one or more average signal values into a signal data flow being processed in substitution for signal values which include signal artefacts arising from the one or more electronic devices coupled to the person.

More optionally, in the monitoring apparatus, the signal processing arrangement is operable to detect a rate of change of signal in the signal data flow, and to perform a substitution for signal values when the rate of change of signal exceeds a threshold value.

Yet more optionally, in the monitoring apparatus, the signal processing arrangement includes a FIFO buffer through which data words corresponding to one or more signals derived from the one or more electrodes are passed in operation, wherein the FIFO buffer is configured for implementing a filter for detecting and removing signal artefacts arising from the one or more electronic devices coupled to the person.

Yet more optionally, in the monitoring apparatus, the signal processing arrangement is operable to store a plurality of samples of non-filtered data and a plurality of samples of filtered data therein for computation of the one or more average signal values, and for detecting the signal artefacts arising from the one or more electronic devices coupled to the person.

Yet more optionally, in the monitoring apparatus, the signal processing arrangement includes an ECG amplifier for amplifying signals from the one or more electrodes to generate corresponding amplified signals, an anti-aliasing filter for bandwidth-limiting the corresponding amplified signals to generate filtered amplified signals, and an analog-to-digital converter for converting the filtered amplified signals into corresponding data words, and the FIFO buffer is operable to receive the data words, wherein the FIFO buffer is arranged to include a series of data words (for example: ADₙ, ADₙ₋₁, ADₙ₋₂, ADₙ₋₃, ADₙ₋₄) and series of filtered values (for example: yₙ₋₅, yₙ₋₆, yₙ₋₇, yₙ₋₈, yₙ₋₉) derived from an output from the FIFO buffer, and processing hardware for executing computations for generating the corresponding processed signals for the display arrangement.

Yet more optionally, in the monitoring apparatus, the processing hardware is operable to compute at least one average value (AVG) from a combination of signal data words and filtered data values (for example: AVG=(ADₙ+ADₙ₋₁+yₙ₋₇+yₙ₋₈+yₙ₋₉)/5), and is operable to compute a baseline (B) from a combination of signal data words and filtered data values (for example: B=minimum [ADₙ, ADₙ₋₁, yₙ₋₇, yₙ₋₈, yₙ₋₉]), and the detection of the signal artefacts arising from one or more electronic devices coupled to the person is performed by comparing the baseline (B), and the at least one average value (AVG) with a signal data word of the FIFO buffer (for example: if (ADₙ₋₄−B)>6*(AVG−B), then ADₙ₋₄ is replaced by AVG, and newer signal values (ADₙ₋₃, ADₙ₋₂) and previously filtered ones (yₙ₋₅, yₙ₋₆) are replaced by AVG).

Optionally, the monitoring apparatus is implemented as a first housing for attachment to a chest region of the person and a second housing for attachment to a portion of the person which is accessible for remote inspection, wherein the one or more electrodes are associated with the first housing, and the display arrangement is associated with the second housing, and wherein the first and second housings are mutually couplable in communication when in operation.

Optionally, in the monitoring apparatus, the display arrangement is operable to provide a visual and/or audible indication of heart beat rate, wherein a rate of blinking and/or a frequency of tone is employed to indicate a measure of the heart beat rate.

Optionally, the monitoring apparatus includes a wireless interface coupled to the processing arrangement for conveying heart beat rate data output from the processing arrangement to a remote location.

More optionally, in the monitoring apparatus, the wireless interface is operable to enable the apparatus to communicate with at least one of: a data recording device, a computer, a laptop computer, a tablet computer, a mobile phone, a Personal Digital Assistant (PDA), a smartphone.

Optionally, in the monitoring apparatus, signals from the one or more electrodes are representative of body surface mapping.

Optionally, the apparatus is packaged and designed to be a once-off use disposable device.

According to a second aspect of the invention, there is provided a method of using a monitoring apparatus for monitoring heart activity by sensing one or more signals accessible at an outer skin surface of a person, wherein the monitoring apparatus includes one or more electrodes for contacting onto the outer skin surface of the person, a signal processing arrangement for processing one or more signals provided from the one or more electrodes to generate corresponding processed signals, and a display arrangement for receiving the processed signals to generate presentation information from which a heart beat rate of the person is discernible, characterized in that the method includes employing in the signal processing arrangement a detector for detecting signal artefacts arising from one or more electronic devices coupled to the person and for removing an influence of the signal artefacts from the processed signals, such that the processed signals provide a more accurate indication of the heart beat rate.

Optionally, the method includes using the signal processing arrangement to remove signal artefacts arising from the one or more electronic devices coupled to the person being a pacemaker.

More optionally, the method includes employing the signal processing arrangement to insert one or more average signal values into a signal data flow being processed in substitution for signal values which include signal artefacts arising from the one or more electronic devices coupled to the person.

More optionally, the method includes employing the signal processing arrangement to detect a rate of change of signal in the signal data flow, and to perform a substitution for signal values when the rate of change of signal exceeds a threshold value.

Yet more optionally, the method includes passing through a FIFO buffer of the signal processing arrangement data words corresponding to one or more signals derived from the one or more electrodes, wherein the FIFO buffer is configured for implementing a filter for detecting and removing signal artefacts arising from the one or more electronic devices coupled to the person.

Yet more optionally, the method includes employing the signal processing arrangement to store a plurality of samples of non-filtered data and a plurality of samples of filtered data therein for computation of the one or more average signal values, and for detecting the signal artefacts arising from the one or more electronic devices coupled to the person.

Yet more optionally, the method is implemented such that the signal processing arrangement includes an ECG amplifier for amplifying signals from the one or more electrodes to generate corresponding amplified signals, an anti-aliasing filter for bandwidth-limiting the corresponding amplified signals to generate filtered amplified signals, and an analog-to-digital converter for converting the filtered amplified signals into corresponding data words, and the FIFO buffer is operable to receive the data words, wherein the FIFO buffer is arranged to include a series of data words (for example: ADₙ, ADₙ₋₁, ADₙ₋₂, ADₙ₋₃, ADₙ₋₄) and series of filtered values (for example: yₙ₋₅, yₙ₋₆, yₙ₋₇, yₙ₋₈, yₙ₋₉) derived from an output from the FIFO buffer, and processing hardware for executing computations for generating the corresponding processed signals for the display arrangement.

Yet more optionally, the method is implemented such that the processing hardware is operable to compute at least one average value (AVG) from a combination of signal data words and filtered data values (for example: AVG=(ADₙ+ADₙ₋₁+yₙ₋₇+yₙ₋₈+yₙ₋₉)/5), and is operable to compute a baseline (B) from a combination of signal data words and filtered data values (for example: B=minimum [ADₙ, ADₙ₋₁, yₙ₋₇, yₙ₋₈, yₙ₋₉]), and the detection of the signal artefacts arising from one or more electronic devices coupled to the person (100) is performed by comparing the baseline (B), and the at least one average value (AVG) with a signal data word of the FIFO buffer (for example: if (ADₙ₋₄−B)>6*(AVG−B), then ADₙ₋₄ is replaced by AVG, and newer signal values (ADₙ₋₃, ADₙ₋₂) and previously filtered ones (yₙ₋₅, yₙ₋₆) are replaced by AVG).

Optionally, the method includes implementing the apparatus as a first housing for attachment to a chest region of the person and a second housing for attachment to a portion of the person which is accessible for remote inspection, wherein the one or more electrodes are associated with the first housing, and the display arrangement is associated with the second housing, and wherein the first and second housings are mutually couplable in communication when in operation.

Optionally, the method includes using the display arrangement to provide a visual and/or audible indication of heart beat rate, wherein a rate of blinking and/or a frequency of tone is employed to indicate a measure of the heart beat rate.

Optionally, the method includes using a wireless interface of the apparatus coupled to the processing arrangement for conveying heart beat rate data output from the processing arrangement to a remote location.

Yet more optionally, the method is implemented such that the wireless interface is operable to enable the apparatus to communicate with at least one of: a data recording device, a computer, a laptop computer, a tablet computer, a mobile phone, a Personal Digital Assistant, a smartphone.

Optionally, the method includes arranging for signals from the one or more electrodes to be representative of body surface mapping.

Optionally, the method includes arranging for the apparatus to be packaged and designed to be a once-off use disposable device.

According to a third aspect of the invention, there is provided a software product recorded on machine-readable data storage media, wherein the software product is executable upon computing hardware for implementing a filter for distinguishing biological signals from a biological signal source from interfering signal artefacts arising from one or more electronic devices employed in conjunction with the biological signal source.

Optionally, the software product is executable for implementing a method pursuant to the second aspect of the invention.

It will be appreciated that features of the invention are susceptible to being combined in various combinations without departing from the scope of the invention as defined by the appended claims.

### DESCRIPTION OF THE DIAGRAMS

Embodiments of the present invention will now be described, by way of example only, with reference to the following diagrams wherein:

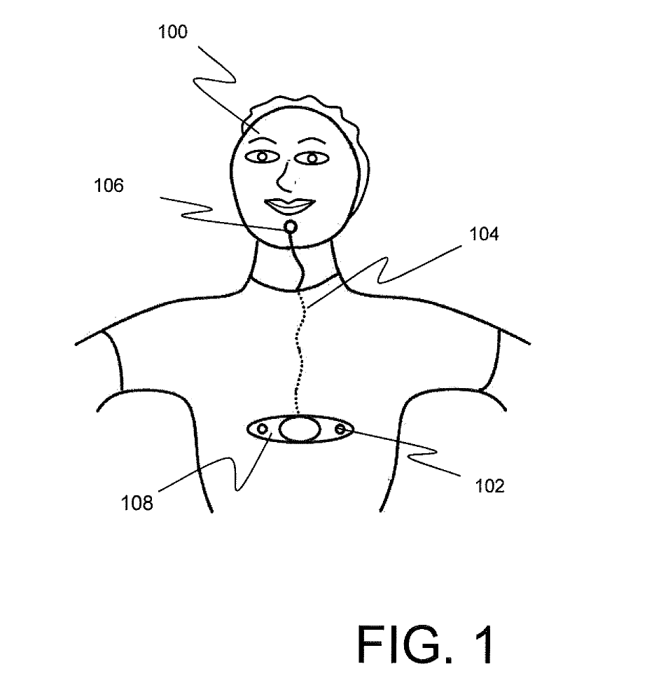

FIG. 1 is an illustration of a monitoring apparatus, referred to as a “Spektikor DHBI-1” apparatus, for providing an ECG of a patient. The apparatus comprises pairs of electrodes (leads) for attaching to the patient, and a signal acquisition and digital filtering unit which is attached in operation with an adhesive backing strip to a ribcage of the patient. The pairs of electrodes provide in use a measured electronic signal which is processed within the apparatus to generate an output signal which is relayed via a wire to a LED display which is placed in use for convenient visualisation by a healthcare professional, for example on a chin of the patient as shown in FIG. 1;

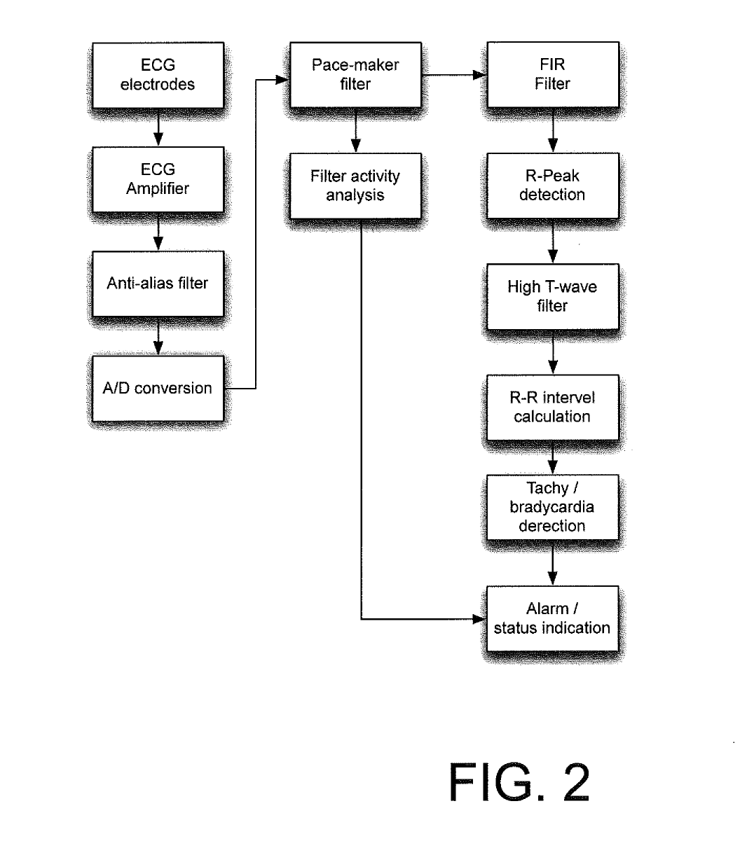

FIG. 2 is a block diagram of signal processing steps S1 to S11 executed within the apparatus of FIG. 1 for removing an artificial pacemaker signal component from the measured electronic signals; the steps S1 to S11 are described in greater detail later;

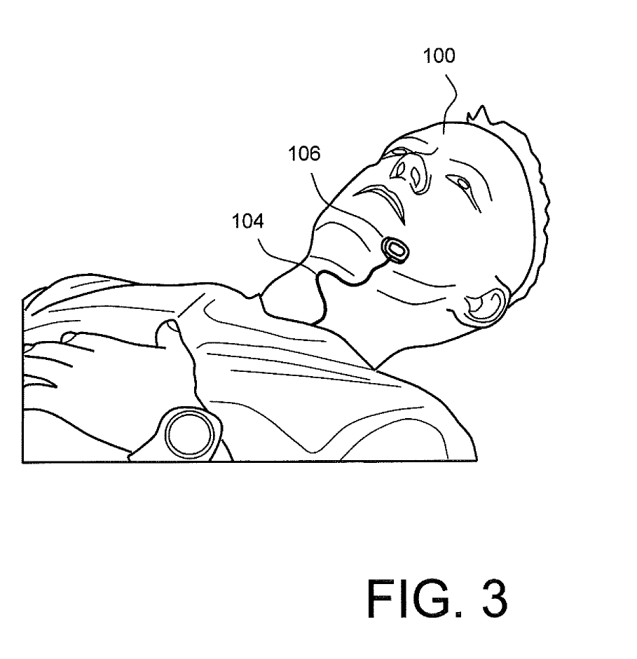

FIG. 3 is an illustration of an injured patient to whom the apparatus of FIG. 1 has been installed; and

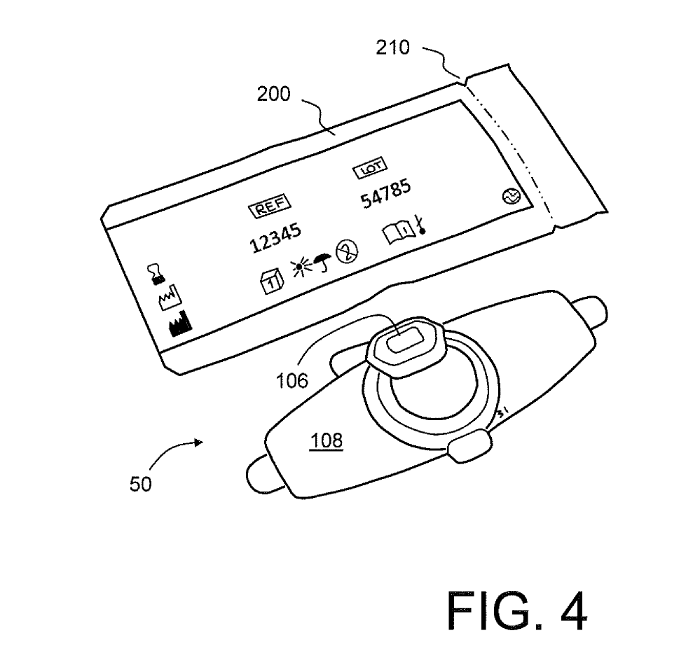

FIG. 4 is an illustration of the sales package for the apparatus of FIG. 1 together with a practical embodiment of the apparatus as described below. The apparatus includes a LED display in a second housing which is positioned remotely from a first housing of the apparatus, for example the second housing is positioned onto a chin of the patient. The apparatus additionally includes a battery for providing power to the apparatus, and a small digital display for enabling direct reading of monitored heart rate

In the accompanying diagrams, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DESCRIPTION OF EMBODIMENTS OF THE INVENTION

In overview, the present invention is concerned with a monitoring apparatus indicated generally by 50 in FIG. 4, namely a medical apparatus 50 for detecting heartbeat. Optionally, the apparatus 50 is disposable after use. The monitoring apparatus 50 includes a digital filter for removing interfering artefacts in sensed heart signals resulting from a presence of electronic devices such as pacemakers. The apparatus 50 enables a more reliable indication of heartbeat to be obtained.

Referring to FIG. 1, there is shown a patient 100 requiring heart beat rate monitoring by using an apparatus 50 pursuant to the present invention. The apparatus 50, for example known as a “Spectrikor DHBI-1” apparatus, comprises first and second housings 108, 106 respectively. The first housing 108 includes electrodes for contacting onto an outer skin surface of the patient 100, a battery and a small digital display backed with an adhesive strip 102. The first housing 108 is coupled via wires 104 to the second housing 106 which includes light emitting diodes, namely LEDs. The first housing 108, via its adhesive strip 102, is attached directly to the outer skin surface of the patient 100, namely to a rib cage region of the patient 100. Moreover, via an adhesive backing strip (not shown), the second housing 106 including the LEDs is attached to a region of the patient 100 which is easily visualised by personnel assisting the patient; for example, the second housing 106 is shown here affixed to a chin of the patient 100. A problem which is potentially encountered when employing the apparatus 50 is that the patient 100 may have one or more electronic devices on or in their bodies, for example a cardiac pacemaker. The pacemaker's purpose is to send pulses to a heart of the patient 100 to regulate a beat of the heart. In the absence of an appropriate digital filter, these pulses may potentially provide a false indication in the apparatus 50. In one embodiment of the invention, within the electrode-containing portion of the apparatus 50, there is included a digital filer that removes interfering signals caused by the presence of an electronic device such as a pacemaker installed in or on the patient 100.

Most commonly, the apparatus of FIG. 1 is attached directly to the outer skin surface of the patient 100, for example over a centre of the rib cage region as shown in FIG. 1. The apparatus 50 may alternatively be positioned to one side or above or below the position shown, for example if the patient 100 has sustained injuries that limit the positioning of the apparatus 50. Yet alternatively, the apparatus 50 may be positioned on a back region of the patient 100.

Although use of the apparatus 50 of FIG. 1 to remove signal artefacts caused by pacemakers is mentioned in the foregoing, it will be appreciated that the apparatus 50 is also capable of removing interfering signal artefacts from other types of electronic devices, for example arising from electrodes implanted into a brain of the patient 100, for example for controlling headaches and/or epilepsy, or electrodes used for controlling the release of medications, for example implanted insulin pumps, or electrodes that are used to address problems associated with damaged nerves, for example in relation to bionic prostheses.

The apparatus 50 of FIG. 1 is of benefit in that it can be deployed extremely rapidly, for example in an event that the patient 100 being found to require emergency assistance. Moreover, the apparatus 50 of FIG. 1 is capable of providing more accurate measurements of heart function in comparison to known monitoring apparatus, for example in situations where the patient 100 has a pacemaker or similar electronic device fitted.

Referring next to FIG. 2, there is shown a block diagram of steps executed within signal processing hardware and/or computing hardware included in the apparatus 50. The apparatus 50 is operable to detect and filter interfering signals from a pacemaker or similar in steps S1 to S11 as provided in Table 1.

TABLE 1
Steps executed within the apparatus 50 of FIG. 1

| Step | Function |
|---|---|
| S1 | ECG electrodes are used to measure signals from the heart of the patient 100. |
| S2 | ECG amplifier amplifies the signal from step S1 |
| S3 | An anti-alias filter is used to filter the amplified signal from step S2 to |
| | correspond to desired sampling rate (typically 500 Hz) to give a filtered analog signal. |
| S4 | The analog signal from step S3 is analog-to-digital converted (ADC) using |
| | an analog-to-digital converter to generate a corresponding digital signal. |
| S5 | The digital signal is subject to pacemaker signal filtering as will be |
| | elucidated in greater detail later. |
| S6 | Filtered data from step S5 is passed to a finite impulse response filter (FIR). |
| | The FIR filter is employed to filter low frequency components away arising, |
| | for example, from movement of the patient 100; the FIR filter is also |
| | employed to filter high frequency components away, for example |
| | disturbances from electrical devices associated with the patient 100. |
| S7 | Filtered ECG measurement data from step S6 is further processed in an R- |
| | peak detector, for detecting an R peak from a QRX complex of the ECG measurement data. |
| S8 | Output from step S7, namely R-peak detector, is followed by further signal |
| | processing in a high T-wave filter. |
| S9 | A R-R calculation block its employed to determine a time interval between |
| | R peaks; in practice this is the pulse of the patient 100. |
| S10 | A tachy/bradycardia block includes set limits for the heart rate determined |
| | in step S9. An upper threshold (tachycardia) for such limits can be preset or |
| | can be adjusted depending on the patient 100 and a situation of the patient |
| | 100. A lower threshold (bradycardia) can be preset or can be adjusted |
| | depending on the patient 100 and a situation of the patient 100, for |
| | example in a range of 50 to 60 beats per minute. |
| S11 | An alarm/Status indicator block uses input from the tachy/bradycardia |
| | block of step S10, and from a filter activity analysis block to indicate to the |
| | healthcare professionals, for example medical doctors and/or other |
| | emergency personnel, the status of the patient 100. |

Step S5 will be described in greater detail on account of its role in implementing the present invention. The ADC in step S4 generates data words which represent the filtered analog signal provided to the ADC The data words are passed along a first-in first-out shift (FIFO) buffer which is operable to function as a filter for removing artefacts arising from pacemakers and similar electronic devices; the filter is complex in its operation and is not merely a simple frequency filter. For example, a pacemaker signal has a duration of 1 milliSecond maximum and has an example amplitude of 0.2 Volt to 0.7 Volt as represented in the data words passing through the FIFO. When implementing the present invention, it is desirable that such filtering to remove pacemaker artefacts is performed early in the steps S1 to S11, and preferably before any bandpass filtering is applied as aforementioned.

In step S5, signal filtering to remove pacemaker artefacts is executed in FIFO, for example implemented as a 10-word length buffer. Data passing through the FIFO are conveniently defined as follows:

TABLE 2
FIFO data structure

Most
recent

| | | | | | | | | | |
|---|---|---|---|---|---|---|---|---|---|
| ADₙ | ADₙ₋₁ | ADₙ₋₂ | ADₙ₋₃ | ADₙ₋₄ | yₙ₋₅ | yₙ₋₆ | yₙ₋₇ | yₙ₋₈ | yₙ₋₉ |

wherein
ADᵢ=an ADC conversion result; and
yᵢ=a previously filtered value at a time i.

In a next phase, an average level of the detected signal is computed pursuant to Equation 1 (Eq. 1) as follows:

$$AVG = \frac{( {AD}_{n} + {AD}_{n - 1} + {y}_{n - 7} + {y}_{n - 8} + {y}_{n - 9} )}{5} \qquad Eq . 1$$

After executing Equation 1 (Eq. 1), a signal baseline B is computed pursuant to Equation 2 (Eq. 2) as follows:

B=minimum[ADₙ,ADₙ₋₂,yₙ₋₇,yₙ₋₈,yₙ₋₉] Eq. 2

The digital filter than analyzes the signal in the FIFO for a time (n−4) as follows:

If (ADₙ₋₄−B)>6*(AVG−B),then the value of ADₙ₋₄ is replaced with the computed value of AVG from Equation 1 (Eq. 1). Eq. 3A

Moreover,signal values ADₙ₋₃,ADₙ₋₂ and previously filtered values for yₙ₋₅,yₙ₋₆ are also replaced. Eq. 3B

Various alternative embodiments of the apparatus 50 will now be described.

In the embodiment described in the foregoing, the filter includes in its FIFO four samples of non-filtered sampled data, and four samples of filtered data. If a most recently received sample received from the analog-to-digital converter (ADC), namely ADₙ₋₄, deviates above a threshold margin from the baseline B, such deviation is indicative that a pacemaker filter is to be utilized. In other words, the filter of the apparatus 50 is operable to switch dynamically between different filtering regimes depending upon temporally sudden changes occurring in the digitized signal being passed through the FIFO. Optionally, a visual indication that a switch between different filtering regimes has been triggered in the apparatus 50, namely a side-step 5.1, is provided; for example, the indication is provided via an illuminated LED included in the second housing 106. Optionally, the pacemaker filter is implemented by signal values corresponding to an excessive signal excursion caused by pacemaker pulsing being replaced by average values as computed using Equation 1 (Eq. 1). Optionally, if signal analysis executed within the apparatus 50 indicates that there are no signal artefacts arising from an electronic device, for example a pacemaker, since

(ADₙ₋₄−B)<6*(AVG−B)

indicating that there are no electronic device artefacts which are adversely influencing pulse rate measurement accuracy, no modifications are carried out on the data words passing through the FIFO pursuant to Eq. 1, Eq. 2, Eq. 3A and Eq. 3B.

Conveniently, in an example embodiment of the apparatus 50, light emitting diodes (LED) are employed to provide measurement indications, for example on the second housing 106, with LED colours employed to denote following conditions:

TABLE 3
LED indications for the apparatus 50.

| LED colour | Green | Orange | Red |
|---|---|---|---|
| Indication | “Heart rate | “Patient 100 has a | “Emergency, |
| | acceptable” | pacemaker” | something wrong” |

Optionally, the LED's are intermittently energized so as to appear flashing to enhance their visibility. More optionally, the LED's are flashed at a temporal rate which is a function of a measured heart beat rate of the patient 100. Alternatively, or additionally, the apparatus 50 provides an audible output when in operation measuring the heart rate of the patient 100, for example one or more types of bleep. For example, the bleep comprises a first reference tone bleep indicative of a reference heart beat rate (for example at a frequency of 440 Hz indicative of a heart beat rate of sixty beats per minute), a second measurement tone bleep whose frequency is a function of the measured heart beat rate of the patient 100 (wherein a measured heart beat rate of sixty beats per minute results in the second tone bleep being at a similar frequency to the first tone bleep) followed by a pause period devoid of any bleep. A succession of the first bleep tone, the second bleep tone and the pause period can be executed in a repetitive cyclical manner and is readily interpretable by trained personnel, for example nursing staff. Optionally, the second tone bleep is of increasing frequency as the measured heart beat rate of the patient 100 increases.

Optionally, the aforementioned tone bleeps are selectively not employed when the signal being filtered in the aforesaid FIFO indicates that no action is required to assist the patient 100, and selectively employed when the patient 100 is found to have a pacemaker fitted, or that the signal being filtered via the FIFO is not acceptable for one or more reasons, for example amplitude is too low, an absence of measurable heat beat, a highly irregular heart beat rate, and so forth.

Optionally, the apparatus 50 includes a wireless interface for providing a representation of the heart beat of the patient 100 to a location which is spatially remote from the apparatus 50. For example, such a wireless interface can be used to provide an alarm to a centralized facility in a hospital in an event of the patient 100 requiring attention from staff at the hospital, for example emergency personnel on site.

In an example embodiment of the apparatus 50, the heart beat signal is acquired from the patient 100 by employing in a range of three to five leads attached to the patient 100 as aforementioned; more optionally, more than five such leads are employed. For example, more than twelve leads are employed for use in implementing body surface mapping in conjunction with the apparatus 50. By “leads”, is meant pairs of contact points to an outer skin surface of the patient 100. Referring to FIG. 3, an example of the patient 100 is shown with the apparatus 50 attached thereto, wherein the first housing 108 is obscured by clothing of the patient 100, and the second housing 106 is visible and attached to a side chin region of the patient 100. The apparatus 50 shown is a “Spektikor DHBI-1” type with its LEDs clearly visible.

Referring next to FIG. 4, there is shown a depiction of the apparatus 50 and its associated sales package 200. The sales package 200 is beneficially hermetically sealed in an inert atmosphere to ensure sterility prior to use of the monitoring apparatus 50 on the patient 100. The sales package 200 includes one or more side notches 210 which allow the package 200 to be opened rapidly when required by applying finger force and/or a cutting tool.

In FIG. 4, there is depicted the apparatus 50 implemented as a “Spectrikor DHBI-1” device. The second housing 106 of the apparatus 50 has LED's which can be withdrawn from the device, wherein its wires are of sufficient length to reach the chin region of the patient 100 or an alternative clearly visible place on a body of the patient 100. The device also includes a battery for providing operating power to the device, and a small digital display whereat heart beat rates can be observed by nursing personnel and similar.

Use of the apparatus 50 will now be described. During deployment, with reference to FIG. 4, the device is unpacked from its package 200, namely from its disposable pouch. Next, electrodes of the device are attached to the patient 100, for example to substantially a centre of the rib cage of the patient 100 by using the adhesive strip 102. Thereafter, the LED's in the second housing 106 are stuck to the patient 100 at a location whereat the LED's are visible to personnel. If heart beat data is to be recorded from the device, a data connection is established between the device and a recording unit, for example a computer, data logger or printer. Heart beat rate is then monitored via inspection of the LED's or remotely, and treatment administered (if appropriate) depending on information conveyed via the LED's and/or data connection and/or via an audible alarm as aforementioned. Optionally, acquisition of data via the device is initiated by withdrawing the second housing 106 away from the first housing 108; for example, such withdrawal removes a plastics material insulating strip away from batteries of the device so that power is provided from the batteries to the device to cause the device to function as aforementioned. Alternatively, or additionally, the device is energized by way of a switch of the device being changed in state from “OFF” to “ON”, for example by way of finger actuation.

As aforementioned, the apparatus 50 includes computing hardware which is operable to execute one or more software products recorded on machine-readable data storage media. Although such software products are intended for use in the apparatus 50, for example implemented as a “Spektikor DGBI-1” device, the software products are optionally executable in other device, apparatus and systems where removal of transient large-signal excursions are to be suppressed from measurements.

Modifications to embodiments of the invention described in the foregoing are possible without departing from the scope of the invention as defined by the accompanying claims. Expressions such as “including”, “comprising”, “incorporating”, “consisting of”, “have”, “is” used to describe and claim the present invention are intended to be construed in a non-exclusive manner, namely allowing for items, components or elements not explicitly described also to be present. Reference to the singular is also to be construed to relate to the plural. Numerals included within parentheses in the accompanying claims are intended to assist understanding of the claims and should not be construed in any way to limit subject matter claimed by these claims.

We claim:

1. A monitoring apparatus (50) for monitoring heart activity by sensing one or more signals accessible at an outer skin surface of a person (100), wherein the monitoring apparatus (50) includes one or more electrodes for contacting onto the outer skin surface of the person (100), a signal processing arrangement (108) for processing one or more signals provided from the one or more electrodes to generate corresponding processed signals, and a display arrangement (106) for receiving the processed signals to generate presentation information from which a heart beat rate of the person (100) is discernible, characterized in that
the signal processing arrangement includes a detector for detecting signal artefacts arising from one or more electronic devices coupled to the person (100) and for removing an influence of the signal artefacts from the processed signals, such that the processed signals provide a more accurate indication of the heart beat rate.

2. A monitoring apparatus (50) as claimed in claim 1, characterized in that the signal processing arrangement is operable to remove signal artefacts arising from the one or more electronic devices coupled to the person (100) being a pacemaker.

3. A monitoring apparatus (50) as claimed claim 2, characterized in that the signal processing arrangement is operable to insert one or more average signal values into a signal data flow being processed in substitution for signal values which include signal artefacts arising from the one or more electronic devices coupled to the person (100).

4. A monitoring apparatus (50) as claimed claim 3, characterized in that the signal processing arrangement is operable to detect a rate of change of signal in the signal data flow, and to perform a substitution for signal values when the rate of change of signal exceeds a threshold value.

5. A monitoring apparatus (50) as claimed in claim 3, characterized in that the signal processing arrangement includes a FIFO buffer through which data words corresponding to one or more signals derived from the one or more electrodes are passed in operation, wherein the FIFO buffer is configured for implementing a filter for detecting and removing signal artefacts arising from the one or more electronic devices coupled to the person (100).

6. A monitoring apparatus (50) as claimed in claim 5, characterized in that the signal processing arrangement is operable to store a plurality of samples of non-filtered data and a plurality of samples of filtered data therein for computation of the one or more average signal values, and for detecting the signal artefacts arising from the one or more electronic devices coupled to the person (100).

7. A monitoring apparatus (50) as claimed in claim 6, characterized in that the signal processing arrangement includes an ECG amplifier for amplifying signals from the one or more electrodes to generate corresponding amplified signals, an anti-aliasing filter for bandwidth-limiting the corresponding amplified signals to generate filtered amplified signals, and analog-to-digital converter for converting the filtered amplified signals into corresponding data words, and the FIFO buffer is operable to receive the data words, wherein the FIFO buffer is arranged to include a series of data words (Example: AD, ADₙ₋₁, ADₙ₋₂, ADₙ₋₃, ADₙ₋₄) and series of filtered values (Example: yₙ₋₅, yₙ₋₆, yₙ₋₇, yₙ₋₈, yₙ₋₉) derived from an output from the FIFO buffer, and processing hardware for executing computations for generating the corresponding processed signals for the display arrangement.

8. A monitoring apparatus (50) as claimed in claim 7, characterized in that the processing hardware is operable to compute at least one average value (AVG) from a combination of signal data words and filtered data values
(Example: AVG=(ADₙ+ADₙ₋₁+yₙ₋₇+yₙ₋₈+yₙ₋₉)/5),
and is operable to compute a baseline (B) from a combination of signal data words and filtered data values
(Example: B=minimum[ADₙ,ADₙ₋₁,yₙ₋₇,yₙ₋₈,yₙ₋₉]),and
the detection of the signal artefacts arising from one or more electronic devices coupled to the person (100) is performed by comparing the baseline (B), and the at least one average value (AVG) with a signal data word of the FIFO buffer
(Example: if (ADₙ₋₄−B)>6*(AVG−B),then ADₙ₋₄ is replaced by AVG,and newer signal values (ADₙ₋₃,ADₙ₋₂) and previously filtered ones (yₙ₋₅,yₙ₋₆) are replaced by AVG).

9. A monitoring apparatus (50) as claimed in claim 1,
characterized in that the apparatus (50) is implemented as a first housing (108) for attachment to a chest region of the person (100) and a second housing (106) for attachment to a portion of the person (100) which is accessible for remote inspection, wherein the one or more electrodes are associated with the first housing (108), and the display arrangement is associated with the second housing, and wherein the first and second housings (106, 108) are mutually couplable in communication when in operation.

10. A monitoring apparatus (50) as claimed in claim 1,
characterized in that the display arrangement is operable to provide a visual and/or audible indication of heart beat rate, wherein a rate of blinking and/or a frequency of tone is employed to indicate a measure of the heart beat rate.

11. A monitoring apparatus (50) as claimed in claim 1,
characterized in that the apparatus (50) includes a wireless interface coupled to the processing arrangement for conveying heart beat rate data output from the processing arrangement to a remote location.

12. A monitoring apparatus (50) as claimed in claim 10, characterized in that the wireless interface is operable to enable the apparatus (50) to communicate with at least one of: a data recording device, a computer, a laptop computer, a tablet computer, a mobile phone, a Personal Digital Assistant, a smartphone.

13. A monitoring apparatus (50) as claimed in claim 1,
characterized in that signals from the one or more electrodes are representative of body surface mapping.

14. A monitoring apparatus (50) as claimed in claim 1,
characterized in that the apparatus (50) is packaged and designed to be a once-off use disposable device.

15. A method of using a monitoring apparatus (50) for monitoring heart activity by sensing one or more signals accessible at an outer skin surface of a person (100), wherein the monitoring apparatus (50) includes one or more electrodes for contacting onto the outer skin surface of the person (100), a signal processing arrangement (108) for processing one or more signals provided from the one or more electrodes to generate corresponding processed signals, and a display arrangement (106) for receiving the processed signals to generate presentation information from which a heart beat rate of the person (100) is discernible, characterized in that
the method includes employing in the signal processing arrangement a detector for detecting signal artefacts arising from one or more electronic devices coupled to the person (100) and for removing an influence of the signal artefacts from the processed signals, such that the processed signals provide a more accurate indication of the heart beat rate.

16. A method as claimed in claim 15, characterized in that the method includes using the signal processing arrangement to remove signal artefacts arising from the one or more electronic devices coupled to the person (100) being a pacemaker.

17. A method as claimed in claim 16, characterized in that the method includes employing the signal processing arrangement to insert one or more average signal values into a signal data flow being processed in substitution for signal values which include signal artefacts arising from the one or more electronic devices coupled to the person (100).

18. A method as claimed in claim 17, characterized in that the method includes employing the signal processing arrangement to detect a rate of change of signal in the signal data flow, and to perform a substitution for signal values when the rate of change of signal exceeds a threshold value.

19. A method as claimed in claim 18, characterized in that the method includes passing through a FIFO buffer of the signal processing arrangement data words corresponding to one or more signals derived from the one or more electrodes, wherein the FIFO buffer is configured for implementing a filter for detecting and removing signal artefacts arising from the one or more electronic devices coupled to the person (100).

20. A method as claimed in claim 19, characterized in that the method includes employing the signal processing arrangement to store a plurality of samples of non-filtered data and a plurality of samples of filtered data therein for computation of the one or more average signal values, and for detecting the signal artefacts arising from the one or more electronic devices coupled to the person (100).

21. A method as claimed in claim 19, characterized in that the signal processing arrangement includes an ECG amplifier for amplifying signals from the one or more electrodes to generate corresponding amplified signals, an anti-aliasing filter for bandwidth-limiting the corresponding amplified signals to generate filtered amplified signals, and analog-to-digital converter for converting the filtered amplified signals into corresponding data words, and the FIFO buffer is operable to receive the data words, wherein the FIFO buffer is arranged to include a series of data words (Example: ADₙ, ADₙ₋₁, ADₙ₋₂, ADₙ₋₃, ADₙ₋₄) and series of filtered values (Example: yₙ₋₅, yₙ₋₆, yₙ₋₇, yₙ₋₈, yₙ₋₉) derived from an output from the FIFO buffer, and processing hardware for executing computations for generating the corresponding processed signals for the display arrangement.

22. A method as claimed in claim 21, characterized in that the processing hardware is operable to compute at least one average value (AVG) from a combination of signal data words and filtered data values
(Example: AVG=(ADₙ+ADₙ₋₁+yₙ₋₇+yₙ₋₈+yₙ₋₉)/5),and
is operable to compute a baseline (B) from a combination of signal data words and filtered data values
(Example: B=minimum[ADₙ,ADₙ₋₁,yₙ₋₇,yₙ₋₈,yₙ₋₉]),and
the detection of the signal artefacts arising from one or more electronic devices coupled to the person (100) is performed by comparing the baseline (B), and the at least one average value (AVG) with a signal data word of the FIFO buffer
(Example: if (ADₙ₋₄−B)>6*(AVG−B),then ADₙ₋₄ is replaced by AVG,and newer signal values (ADₙ₋₃,ADₙ₋₂) and previously filtered ones (yₙ₋₅,yₙ₋₆) are replaced by AVG).

23. A method as claimed in claim 15, characterized in that the
method includes implementing the apparatus (50) as a first housing (108) for attachment to a chest region of the person (100) and a second housing (106) for attachment to a portion of the person (100) which is accessible for remote inspection, wherein the one or more electrodes are associated with the first housing (108), and the display arrangement is associated with the second housing, and wherein the first and second housings (106, 108) are mutually couplable in communication when in operation.

24. A method as claimed in claim 15, characterized in that the
method includes using the display arrangement to provide a visual and/or audible indication of heart beat rate, wherein a rate of blinking and/or a frequency of tone is employed to indicate a measure of the heart beat rate.

25. A method as claimed in claim 15, characterized in that the
method includes using a wireless interface of the apparatus (50) coupled to the processing arrangement for conveying heart beat rate data output from the processing arrangement to a remote location.

26. A method as claimed in claim 15, characterized in that the
wireless interface is operable to enable the apparatus (50) to communicate with at least one of: a data recording device, a computer, a laptop computer, a tablet computer, a mobile phone, a Personal Digital Assistant, a smartphone.

27. A method as claimed in claim 15, characterized in that the
method includes arranging for signals from the one or more electrodes to be representative of body surface mapping.

28. A method as claimed in claim 15, characterized in that the
method includes arranging for the apparatus (50) to be packaged and designed to be a once-off use disposable device.

29. A software product recorded on machine-readable data storage media, wherein the software product is executable upon computing hardware for implementing a filter for distinguishing biological signals from a biological signal source (100) from interfering signal artefacts arising from one or more electronic devices employed in conjunction with the biological signal source (100).

30. A software product as claimed in claim 29, characterized in that the software product is executable for implementing a method
of using a monitoring apparatus (50) for monitoring heart activity by sensing one or more signals accessible at an outer skin surface of a person (100), wherein the monitoring apparatus (50) includes one or more electrodes for contacting onto the outer skin surface of the person (100), a signal processing arrangement (108) for processing one or more signals provided from the one or more electrodes to generate corresponding processed signals, and a display arrangement (106) for receiving the processed signals to generate presentation information from which a heart beat rate of the person (100) is discernible, characterized in that the method includes employing in the signal processing arrangement a detector for detecting signal artefacts arising from one or more electronic devices coupled to the person (100) and for removing an influence of the signal artefacts from the processed signals, such that the processed signals provide a more accurate indication of the heart beat rate.
